(19) United States
(12) Patent Application Publication
(10) Pub. No.: US 2025/0320443 A1
(43) Pub. Date: Oct. 16, 2025

(54) Human Brain Organoid, methods for making the brain organoid, and methods of using the brain organoid for high throughput rapid screening and/or evaluation of agents

(71) Applicant: NeyroblastGX LLC, Murrieta, CA (US)

(72) Inventors: Aynun N. Begum, Murrieta, CA (US); Alexander V. Michkov, Murrieta, CA (US)

(21) Appl. No.: 18/980,932

(22) Filed: Dec. 13, 2024

Related U.S. Application Data
(60) Provisional application No. 63/610,746, filed on Dec. 15, 2023.

(51) Int. Cl. C12M 3/00; C12M 1/32; C12M 1/34; C12N 5/079

(52) U.S. Cl. CPC C12M 21/08; C12M 23/12; C12M 41/46; C12N 5/0618; C12N 2502/081; C12N 2503/02; C12N 2506/45; C12N 2513/00

(57) ABSTRACT

This invention relates to a method of generating ready-to-use organoids combined with screening and/or evaluating tools to determine the effects of one or more agents on one or more organs.

### RELATED APPLICATION AND CLAIM OF PRIORITY

This patent application claims priority to, and incorporates by reference, U.S. provisional patent application No. 63/610,746 filed on Dec. 15, 2023, titled Human Brain Organoid, methods for making the brain organoid and methods of using the brain organoid for high throughput rapid screening and/or evaluation of agents.

### FILED OF THE INVENTION

This invention relates to a method of generating ready-to-use organoids combined with screening and/or evaluating tools to determine the effects of one or more agents on one or more organs comprising:
a. applying one or more agents to a highly defined brain organoid combined with a plate comprising one or more electrodes and one or more organoids of the one or more organs;
b. obtaining electrical signals from the ready-to-use organoid electrode plate after the application of one or more agents and
c. process the electrical signals to determine a screening and/or evaluate the result of one or more therapeutics.

### BACKGROUND

Various patents, patent applications, and publications are cited herein, from which these disclosures are incorporated by reference in their entirety. However, the citation of any reference herein should not be construed as an admission that such reference is available as “Prior Art” to the present application.

The brain is the most intricate and complex organ in controlling and maintaining the entire body system. Our current brain or brain-related disorder research is mainly based on animal models and traditional 2D cell cultures.¹ However, the inherent species differences between humans and animals and cytoarchitectural dissimilarities between 2D cellular and 3D organ models cannot accurately recapitulate human brain development and associated disorders.^{1, 2, 3} Moreover, most patient studies do not capture the earliest stages of disease development, thus unable to provide a complete profile of mechanistic insights into the disease.^{1, 2} In this context, human pluripotent stem cell-derived brain organoids, such as cerebral^{4, 5, 6} and choroid plexus^{7, 8} organoids, have been reported to recapitulate many key features of the human brain in vivo, for example, secreting cerebrospinal fluids (CSF) or brain CSF barrier to study drug permeability, availability, and toxicity. Moreover, these brain organoids are not limited to screening drugs or drug-related toxicity. Brain organoids (OrG) s can also test disease-related toxic proteins, chemicals, inhibitors, toxins, and pathogens such as Zika or COVID-19 viral infections linked to neurodevelopmental or neurodegenerative disorders. In this context, NeyroblastGX LLC (NGL) developed OrGspheroid™ to generate time-dependent cerebral organoids integrating microglia, blood-brain barrier microvascular endothelial cells (MVECs), and further generation of choroid plexus containing brain CSF barrier epithelium to determine and monitor various agent causing disease or toxicity using high-throughput sensing arrays. We have produced a method for ready-to-use complex human brain OrG and various other OrGs grown on a high-throughput (HT) electroconductive sensing array to rapidly screen agents for diagnostic and therapeutic purposes.

NGL's brain OrG developed from human induced pluripotent stem cells (HiPSCs)-derived OrGspheroids co-culture with microglia and MVECs, which led to the developing of a tri-layer culture that can mimic a complex central nervous system (CNS) to study brain disorders via screening pathogenic proteins or any agents. Our defined OrGspheroid's microenvironment is highly suitable for integrating various CNS cells, such as pericytes and oligodendrocytes, not limited to microglia or MvECs. Further, this tri-layer culture can be more precisely grown on a 96-well electrode plate with ˜5-mm-deep grooves in each well that can hold the brain OrG in a defined geometrical position. The grooving pattern in the bottom and top electrodes of each well ensures no movement of the floating brain and produces a reproducible and sensitive electrical signal designed and developed by NGL (FIG. 7A-7D). Besides, we have already tested our complex brain OrG with the electric cell-substrate impedance sensor (ECIS) in collaboration with Applied Biophysics, Inc. (ABP) and obtained electrical signals with synthetic amyloid-beta (AB) and tau oligomers to recapitulate the synaptic loss (FIG. 8B-8D) usually observed in late onset of Alzheimer's disease (LOAD)^{9, 10}. Thus, our novel brain organoids are ideal for ECIS or any commercially available electrode to provide a ready-to-use CNS microphysiological system (MPS) for HT screening tool for an agent to accelerate the development of new biomarkers and therapeutics to cure or prevent brain and related disorders. No electrode plate has been created for floating brain OrG or any other OrGs or 3D spheroids that can accurately recapitulate human brain physiology, which NGL has designed and created for the novel scalable CNS brain OrG. Thus, our brain OrG with tri-layering of CNS cells led to obtaining glutamatergic neurons, glial cells, and BBB cells with proper positioning in the electrode, will have higher sensitivity and specificity to capture the electric signal derived from the entire brain with drugs or toxic proteins or any agents. These electrical signals will correlate with pathological biomarkers of brain disorders, such as inflammatory cytokines or pathogenic oligomeric proteins and gene expression changes, which bring novelty to our ready-to-use HT technology for testing and screening compared to traditional 2D cell culture or animal models. Therefore, customers can buy defined human brain OrG optimized with our custom-made electrode plate compatible with commercially available electrode measurement units to screen preclinical drugs and toxicity rapidly and sensitively without batch-to-batch variation, providing a rapid and novel microphysiological system (MPS) for testing or screening agents.

### SUMMARY OF THE INVENTION

NGL-generated brain OrG from HiPSC-derived OrGspheroids™, a new and novel condition for brain OrG development. Our brain OrG microenvironments condition, such as media, chemicals, and growth factors, leads to the generation of corticosphere (Csphere) fully functional mature cerebral organoids by four weeks (28 days). In 28 days, we integrated microglial and MVECs to recapitulate CNS cerebral with blood-brain barrier (BBB) with higher efficiency. OrGspheroid is also highly efficient in developing choroid plexus (ChP) organoids containing secretory epithelium of ChP to actively secrete CSF and forming the blood CSF barrier over five weeks (40 days) with ChP cocktails (neural condition media combination with serum replacement supplements). Thus, our HiPSC-derived OrGspheroids are highly efficient in producing high-quality scalable OrGs like cerebral and ChP OrGs.

OrGspheroid condition in gel matrix domes with our complete neural growth media component promotes the generation of early cortical neural progenitor markers by day 17, expressing SOX2⁺, β, III Tubulin⁺, PAX6⁺, ZO1⁺, Reelin⁺, TBR1⁺, TBR2⁺, SATB2⁺, MAP2⁺ and Vglut1⁺ marker expressions. We have also integrated microglial types cells into cortical neural progenitor cells during this stage. Microglia integration with cortical neurons didn't interfere with their marker cell expression.

On day 17, early cerebral OrG was collected and further added MvECs and brought to the culture using gel matrix domes for three days, additionally released gel matrix domes into floating media and maintained with an orbital shaker to produce mature CNS cerebral (cCerebral) OrG up to ˜3 mm by 28 days with robust expression of cortical neuronal markers (MAP2⁺, β III Tubulin⁺ or TUJ1⁺, SATB2⁺, FOXP2⁺, BRN2⁺, TBR1⁺), glial markers and microvasculatures cytoarchitecture (IBA1⁺, GFAP⁺, and CD144 or VE-Cadherin), excitatory synaptic and glutamatergic neuronal markers (PSD-95⁺, Synaptophysin⁺, Vglut1⁺). Our brain OrG in a gel matrix dome efficiently integrates microglia and MVECs without interfering with the outcome of various cortical layer neurons. Thus, brain OrG can be used for screening agents involved in or preventing CNS disorders.

OrGspheroid with ChP containing 40% neural condition media combined with 60% serum replacement cocktail with longer culture, we could generate choroid plexus (ChP) OrG within 40 days. ChP OrG generation takes over five weeks to produce a cuboidal cell monolayer containing ChP with a fluid-filled cavity that can actively produce CSF with the expression of Aquaporin 1⁺, Prealbumin⁺, and VE-Cadherin⁺, ZO1⁺ marker cells, which usually express in human ChP or CSF. We also observed typical myoepithelial cells, a cell type in the secretory glands of ChP, as shown via Giemsa staining.

We have prepared a polyphenolic agent (compound) curcumin, formulated it using solid lipid curcumin nanoparticles (NP), and tested it on ChP OrG culture. Curcumin (Cur) is an antioxidant and neuroprotective compound that disaggregates amyloid-beta (AB), a toxic protein contributing to Alzheimer's disease (AD) pathology. Traditionally, Cur is ingested with water, which cannot dissolve, resulting in no bioavailability and efficacy in various clinical trials. We aim to see if our ChP model can differentiate formulated and unformulated curcumin bioavailability status. We have found that formulated Cur was permeable into CSF up to 16 μM, but unformulated Cur was aggregated and unable to pass the blood CSF barrier and remains undetected in CSF. Thus, ChP and our electrode plate combined MPS can be efficient HT tools for testing drugs or any agents to see brain toxicity or pathology more precisely.

To screen our brain OrG, we have developed an HT electroconductive plate that allows the growth of floating brain OrGs to determine the entire brain's electrical signal at various excitation frequencies. The well plate has a typical 8×12 configuration that allows 96 samples to be measured, upgraded to 384 wells and is compatible with floating brain OrG. This is the first novel approach for generating electrode plates for floating brain OrG or any OrG or 3D spheroids to control OrG size without having batch-to-batch variation and obtain electrical signal reproducibility and sensitivity with higher specificity. Moreover, this HT electrode plate provides electrical signals from the entire brain network rather than selective neurons.

We have also tested our brain OrG with other commercially available Electric Cell-substrate Impedance Sensing (ECIS) electrode plates and measuring units [(Applied Biophysics Inc (ABP)] to understand whether our ready-to-use brain OrG is compatible with other commercially available electrodes. We have grown our cCerebral OrG by attaching it to ABP's ECIS plate using gel-matrix domes to do this. ABP or other commercially available electrode plates are designed to attach OrGs into the plate via gel-matrix domes and measure electrical current. This can also be done using NGL ready-to-use and scalable brain OrG (cCerebral and ChP OrG). ABP's electrode plate-grown NGL cCerebral OrG was tested with AD toxic agents (AB and tau) via injection and evaluated using immunocytochemistry (ICC) to determine their toxic effect on the synaptic neurons and determined by their markers, such as synaptophysin and PSD-95. We found AD toxic agents induced cCerebral OrG's electrical current, comparable to the synaptic loss marker proteins usually observed in the AD neuronal loss model.

Our novel cCerebral OrG culture was also maintained over 90 days, and no necrotic core was found in this ˜3 mm brain OrG. However, large tissue cultures usually cause necrotic cores^{11, 12} making NGL brain OrG suitable for use in any electrode plate for long-term culture conditions with agents for scalable HT screening of drugs or toxicity. We also used cryopreserved (Cryo) cCerebral OrG, which was thawed and cultured for seven days. We compared their cell viability with freshly cultured and dead cCerebral OrG using a 3-(4, 5-dimethylthiazol-2-yl)-2, 5-diphenyl-2H-tetrazolium bromide (MTT) assay. 13 MTT results confirmed that our Cryo or long culture cCerebral OrG is highly stable and suitable for electrodes for HT screening with agents for understanding disease pathology or accelerating the development of new drugs.

We evaluated that our novel OrGspeheroid and complete neural growth media component can generate highly scalable human cCerebral and ChP OrG with their marker expression, usually observed in human CNS. 4, 5, 6 Our human cCerebral OrG is highly stable and unique to integrate microglia and MVECs to provide complete human CNS mimic cerebral cytoarchitecture without compromising or losing any cerebral marker cells. We also designed and fabricated a novel electrode plate where floating brain OrG can grow and control their size without having any batch-to-batch variation. This electrode plate-grown, ready-to-use floating brain OrG, provides novel MPS for screening agents with higher sensitivity and specificity. Besides our brain OrGs are also compatible with commercially available ABP and other electrode plates to produce sensitive signals correlated with synaptic markers. Moreover, NGL brain OrG can also possibly keep culture over 90 days without having a necrotic core, providing a new tool for disease modeling with various s agents and monitoring biomarkers or disease progression.

### BRIEF DESCRIPTION OF THE DRAWINGS

FIG. 1A describes a time course from day 3 to day 10 for mature CNS cerebral organoids (cCerebral) development.

FIG. 1B describes a time course from day 12 to day 18 for mature CNS cerebral organoids (cCerebral) development.

FIG. 1C describes a time course from day 20 to day 28 for mature CNS cerebral organoids (cCerebral) development.

FIG. 2A describes the morphogenesis of neuroepithelium and radial glial cells with the robust expression of early cortical neurons with the expression of ZO1 and PAX6.

FIG. 2B describes the morphogenesis of neuroepithelium and radial glial cells with the robust expression of early cortical neurons with the expression of B III Tubulin and SOX2.

FIG. 2C describes the morphogenesis of neuroepithelium and radial glial cells with the robust expression of early cortical neurons with the expression of SATB2 and MAP2.

FIG. 2D describes the morphogenesis of neuroepithelium and radial glial cells with the robust expression of early cortical neurons with the expression of B III Tubulin and TBR1.

FIG. 2E describes the morphogenesis of neuroepithelium and radial glial cells with the robust expression of early cortical neurons with the expression of TBR2 and MAP2.

FIG. 2F describes the morphogenesis of neuroepithelium and radial glial cells with the robust expression of early cortical neurons with the expression of Reelin and MAP2.

FIG. 3A describes the formation of mature cerebral integrating CNS glial and microvascular cells for markers MAP2 and GFAP.

FIG. 3B describes the formation of mature cerebral integrating CNS glial and microvascular cells for markers MAP2 and GFAP (insert).

FIG. 3C describes the formation of mature cerebral integrating CNS glial and microvascular cells for marker IBA1.

FIG. 3D describes the formation of mature cerebral integrating CNS glial and microvascular cells for marker β III Tubulin.

FIG. 3E describes the formation of mature cerebral integrating CNS glial and microvascular cells for marker SATB2.

FIG. 3F describes the formation of mature cerebral integrating CNS glial and microvascular cells for marker FOXP2.

FIG. 3G describes the formation of mature cerebral integrating CNS glial and microvascular cells for marker BRN2.

FIG. 3H describes the formation of mature cerebral integrating CNS glial and microvascular cells for marker TBR1.

FIG. 3I describes the formation of mature cerebral integrating CNS glial and microvascular cells for marker ZO1.

FIG. 4A describes the generation of functional glutamatergic excitatory cortical neurons for marker PSD-95.

FIG. 4B describes the generation of functional glutamatergic excitatory cortical neurons for markers β III Tubulin and Vglut1.

FIG. 4C describes the generation of functional glutamatergic excitatory cortical neurons for markers PSD-95 and Vglut1.

FIG. 5A describes the development of reproducible ChP OrG when compared to cCerebral OrG from three different HiPSC lines on day 28 and day 40.

FIG. 5B describes the development of reproducible ChP OrG with Giemsa staining shown in the ChP secretory gland.

FIG. 5C describes the development of reproducible ChP OrG with marker Aquaporin-1 expression.

FIG. 5D describes the development of reproducible ChP OrG with marker Aquaporin-1 expression insert.

FIG. 5E describes the development of reproducible ChP OrG with Pre-Albumin expression.

FIG. 5F describes the development of reproducible ChP OrG with VE-Cadherin expression.

FIG. 5G describes the development of reproducible ChP OrG with ZO1 expression.

FIG. 6A describes the chemical structure for curcumin a natural polyphenolic antioxidant.

FIG. 6B describes the preparation of curcumin with H₂O and lipids.

FIG. 6C describes the solid lipid curcumin particle scanned with scanning electron microscope.

FIG. 6D describes the how ChP OrG efficiently screens drug permeability.

FIG. 6E describes the how ChP OrG efficiently screens drug permeability.

FIG. 6F describes the how ChP OrG efficiently screens drug permeability.

FIG. 6G describes the how ChP OrG efficiently screens drug permeability.

FIG. 6H describes the how ChP OrG efficiently screens drug permeability.

FIG. 7A describes the rapid HT screening electrode plate for floating OrG.

FIG. 7B describes the rapid HT screening electrode plate for floating OrG.

FIG. 7C describes the rapid HT screening electrode plate for floating OrG.

FIG. 7D describes the rapid HT screening electrode plate for floating OrG.

FIG. 7E describes the rapid HT screening electrode plate for floating OrG.

FIG. 8A describes the method to verify compatibility to screening NGL ready-to-use brain OrG in other screening platforms with multiple agents.

FIG. 8B describes the method and results to verify compatibility to screening NGL ready-to-use brain OrG in other screening platforms with multiple agents.

FIG. 8C describes the method and results to verify compatibility to screening NGL ready-to-use brain OrG in other screening platforms with multiple agents.

FIG. 8D describes the method and results to verify compatibility to screening NGL ready-to-use brain OrG in other screening platforms with multiple agents.

FIG. 9A describes the freshly cultured brain OrG versus Cryo brain OrG viability and stability.

FIG. 9B describes the prolonged cultured brain OrG versus Cryo brain OrG viability and stability.

FIG. 9C describes the Cryo cCerebral OrG cultures.

FIG. 9D describes the necrotic cCerebral OrG tissue.

FIG. 9E describes the MTT quantification of cCerebral OrG results.

### DETAILED DESCRIPTION OF THE INVENTION

The present invention will now be described hereinafter with reference to the accompanying drawings and examples, in which embodiments of the invention are shown. This description is not intended to be a detailed catalog of all the different ways the invention may be implemented or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which does not depart from the instant invention. Hence, the following descriptions are intended to illustrate some particular embodiments of the invention and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is to describe particular embodiments only and is not intended to be limiting to the invention.

All publications, patent applications, patents, and other references cited herein are incorporated by reference in their entirety for the teachings relevant to the sentence and paragraph in which the reference is presented.

As used in the description of the invention and the appended claims, the singular forms “a,” “an,” and “the” are intended to include the plural forms as well unless the context indicates otherwise.

The term “organoid” or “organoids” “(OrG or OrGs)” is a singular or plural form used to express due to their use in a certain cell culture environment or to perform a specific assay. OrGs are tiny (mm levels), self-organized, three-dimensional tissue cultures that are derived from pluripotent stem cells, which can be derived from mice, primates, or other human stem cells such as mesenchymal or bone marrow-derived stem cells. But in our case, we developed OrGs from HiPSCs. These human stem cell-based tissue cultures can be created to replicate similar complexity of human organs with a certain type of cells or tissues. Our novelty is that we have generated organoids to recapitulate human organs with collective cell types to perform particular functions like the CNS brain cells. We can control their shape using our HT electrode system in floating conditions and treating them with various agents to develop new diagnostics or therapeutics.

As used herein, “optimized” refers to our organoid or OrG system with the same size grown on the defined geometrical electrode with the same media and floating condition. This will lead us to no batch-to-batch variation. Our brain OrG or OrGs culture system would be our unique, optimized OrG culture development as proposed for screening any agent.

As used herein, the term “cCerebral” or “ChP” refers to the CNS mimic cerebral organoid containing most human CNS cells similar to human CNS in vivo. ChP refers to the choroid plexus of cells arising from the tela choroidea in each of the brain's ventricles. ChP regions produce and secrete most of the CSF of the CNS. Our HiPSCs-derived ChP OrGs have a protective epithelium barrier with fully functional fluid-filled cavities that produce and secrete CSF.

As used herein, “Immunocytochemistry” or “ICC” refers to an assay to determine the presence of a specific cerebral cortex of the various layers, CNS glial, microvascular endothelial, and CSF epithelium or its fluid-based proteins or antigen expression, which is used by various antibodies as discussed in the drawing and findings and visualized by microscope. We have ensured our cCerebral and ChP OrG was viable by determining 4′, 6-diamidino-2-phenylindole (DAPI), a positive area of the OrG marker cell. Our brain OrG upregulated all the functional proteins and expressed robustly. We have used all the CNS's functional proteins to establish that our cCerebral or ChP OrG is fully functional, complete, and mature, similar to human cerebral or ChP in vivo.

As used herein, the terms “High Throughput” or “HT” are methods to describe our “organoid or OrG” system with an electroconductive plate used or can be used to determine various agents with a large number of samples with faster and higher sensitivity. Our electroconductive plate with optimized OrG can screen over 50 agents or drugs in just 3-hour time points and replace the 3-6 months of screening with animals. Using our HT electrode makes it possible to detect entire brain activity rapidly and sensitively rather than selective neurons, which is part of the novelty of our method.

HT electroconductive sensing array combined with our optimized brain OrG is the first innovative technology that can be marketed for floating brain OrG for screening pathogens, drugs, and toxicity at a rapid and robust scale. Brain OrG-specific shape 1-3 mm range grown on an electroconductive plate designed and developed by NGL with pay-based service to obtain ECIS base electrode with a desired signal with or without agents. ECIS here refers to electric cell-substrate impedance sensing. We first developed 8 well plates with an electrode. Once our cCerebral OrG's electrode signal significantly differed from the media, we developed HT-scale 96 well plate with two electrodes to capture the signal better in each cCerebral OrG with agents. We used a nanofarads capacitance (nF) signal curve at 64000 Hz. We determined that the media sample (without OrG, baseline) average signal is 3.7 nF compared to cerebral OrG (˜2 nF), and the same measurement was also acquired by resistance (Ohms). NGL electrode signal in 64000 Hz significantly and sensitively differentiated brain OrG-derived toxic response signal with AB or tau compared to untreated brain OrG.

“Floating OrG” refers to the brain OrG (cCerebral or ChP OrG) grown on the plate in a floating condition using neural media. We have developed a floating brain OrG-grown electrode plate prototype consisting of cylindrical shapes with shallow depth on the bottom up to ˜5 mm where cCerebral OrG can be loaded or grown. This plate can be optimized to 96 and further 384-well plate with an electrical measuring unit, which refers to MSU consisting of ethernet-enabled that controls a direct digital synthesizer to allow excitation signals to be generated with variable frequency and amplitude. This MSU is miniature and can be used and controlled by a portable computer outside the lab to monitor or acquire data measured independently. This brings an easy and rapid system for a quick and affordable HT brain OrG testing platform.

As used herein, “Freshly”, “prolonged,” and “Cryo” refer to cCerebral OrG maintaining in standard culture conditions (37° C. with 5% CO2) are the Freshly cultured cCerebral OrG. But we called it Cryo cCerebral OrG when we stored freshly cultured OrG in cryopreserving media, cooled it in liquid N2 (LN2), and returned it to standard culture condition. Freshly cultured samples were never exposed to cold conditions, but Cryo OrG was exposed once or twice in LN2. Prolonged culture is similar to freshly cultured OrG, never exposed to Cryo condition, but cultured over 90 days in standard culture conditions. We verified Cyro or prolonged (long) OrG cultures are highly viable and stable in the freeze and thaw cycle, similar to freshly cultured OrG, and recapitulate the same human in vivo functionality as the human brain or CNS.

As used herein, “MTT” refers to 3-(4, 5-dimethylthiazol-2-yl)-2, 5-diphenyl tetrazolium bromide which, used for applying to the cCerebral OrG and observed their viability when MTT yellow dye to turn to purple means viable cells with an active metabolism converted MTT (yellow color) into a purple-colored formazan product, which is highly visualized in an MTT solubilized solution that was further measured with a microplate reader with an absorbance near 570 nm.

Dunn's multiple comparisons test is a statistical test that compares the difference in the sum of ranks between two columns with the expected average difference (based on the number of groups and their size). For each pair of columns, GraphPrism (San Diego, CA) reports the P value as >0.05 (not significant) but <0.05 significant, such as *<0.05, **<0.01, ***<0.001, or ****<0.001.

With reference to the Figures of the application:

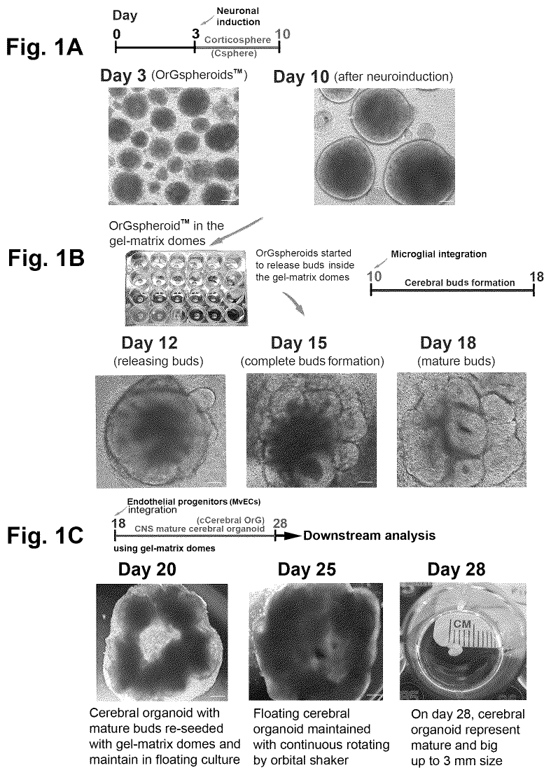

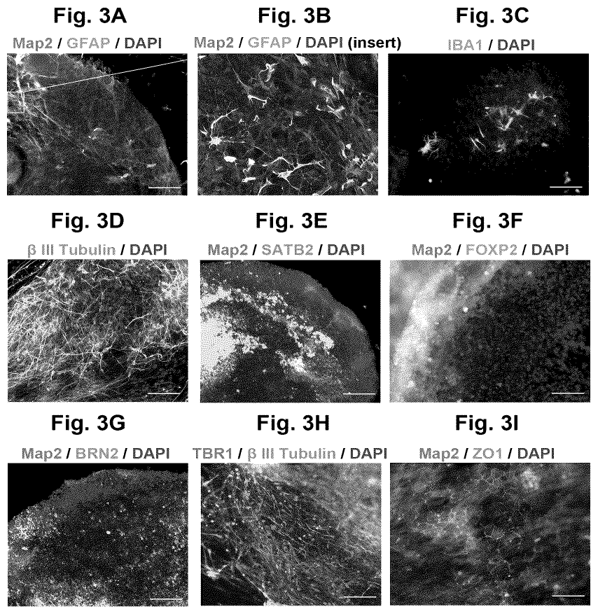

FIG. 1A-1C. A time course of mature CNS cerebral organoids (cCerebral) development. (FIG. 1A) Our method first generates OrGspheroids from HiPSCs at 100-200 μm within the day (D) 3. On D3, OrGspheroids were treated with dual inhibition of SMAD signaling small molecules SB431542 (5 μM) and dorsomorphin (1 UM). On D10, OrGspheroids became big with bright translucent edges and darker colors in the middle (400-500 μm) by treating both small molecules and producing a cortical progenitor-rich corticosphere (Csphere). Scale bar 150 μm. (FIG. 1B) After that, Csphere was seeded in the gel-matrix domes, including added previously prepared microglia, and maintained with complete neural growth media. We found Csphere started to produce buds within two days in the gel-matrix domes. D15 Csphere started to produce large numbers of buds, and D18 buds matured and formed cerebral OrGs. In this stage, cerebral OrG needs to re-seed or cryopreserved. Scale bar 300 μm. (FIG. 1C) To re-seed and generate CNS (c) Cerebral OrG, we culture them on the ultra-low attachment 24-well plates with gel-matrix with microvascular endothelial cells (MVECs). On D20 cCerebral OrG released automatically from the plate attachment and maintained in floating condition with an orbital shaker with continuous rotation. On D28, the cCerebral OrG becomes large with a dark core up to 3 mm, integrating microglia and MvECs with mature cortical neuronal, glial, and microvascular cytoarchitecture visualized, as shown in FIG. 3A-3C. NGL cCerebral OrG integrates microglia and MvECs without affecting the outcome of the cortical layer's neurons. This cCerebral OrG can maintain culture longer than four weeks to appear in microvasculature cytoarchitecture. Our unique and CNS cell-enriched complex cerebral OrG is unique and ready to use for screening drugs, toxicity, chemicals, disease pathological proteins, or underlying disease mechanisms of brain disorders using HT microelectrode plates. Scale bar 500 μm, D; Day.

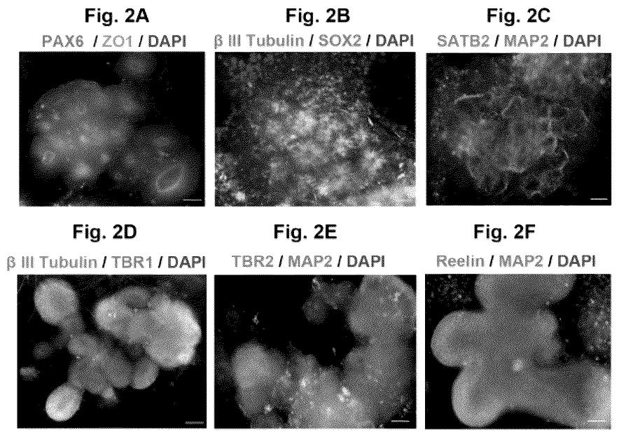

FIG. 2A-2F. Morphogenesis of neuroepithelium and radial glial cells with the robust expression of early cortical neurons. During the appearance of buds in the Csphere, the outcome of the neuroepithelium further maintained the neuroepithelium and generation of early cortical layering cells in the gel-matrix domes with the expression of (FIG. 2A) ZO1 and PAX6, (FIG. 2B) β III Tubulin and SOX2, (FIG. 2C) SATB2 and MAP2, (FIG. 2D) β III Tubulin and TBR1, (FIG. 2E) TBR2 and MAP2, and (FIG. 2F) Reelin and MAP2. We have observed distinct neuroepithelium (ZO1) and SOX2 expression during the formation of neuroepithelium into radial glial cells and further differentiation of ventricular and subventricular zone (SVZ) with expression of early cortical neurons. In early cortical layering, usually appeared PAX6, TBR1, TBR2, Reelin, SATB2, B III Tubulin, and MAP2 in the apical surface in the ventricular zone (VZ), as shown in our culture. Scale bar 150 μm for all the images except images B and F (80 μm). This stage of cortical layering is recapitulated in the developing human brain, which can be studied and screened for neurodevelopmental disorders and their complex nature and new therapeutics.

FIG. 3A-31. Formation of mature cerebral integrating CNS glial and microvascular cells. NGL cerebral OrG was re-seeded and incorporated with microvascular endothelial cells (MvECs) within the gel-matrix domes. This process integrated CNS glial and microvascular cells into cerebral OrG, leading to fully functional CNS cerebral (cCerebral) development within day 28. Our method required a time-dependent increase of our complete neural growth media in culture, capable of generating neuroepithelium, ventricular and subventricular zones (VZ and SVZ), and mature cortical plates. Each stage of cerebral formation, such as neural induction to Csphere to cCerebral, we have used the same neural media, unique to our system, for providing proper nutrition and integrating and maintaining microglia and MVECs without interrupting cortical patterning. Our cCerebral OrG expresses (FIG. 3A-3B) mature neuronal and astrocyte markers (MAP2 and GFAP). Inserts showing visualize dendrites of neurons and spongiform morphology of astrocytes. (FIG. 3C) Integrating microglia with microglial marker expression (IBA1), (FIG. 3D) neuron-specific marker (III Tubulin, (FIG. 3E) late born superficial cortical layer marker (SATB2), (FIG. 3F) glutamatergic deep layer cortical marker (FOXP2), (FIG. 3G) cortical layers 2-3 marker cells BRN2, and (FIG. 3H) cortical preplate to all the cortical layers marker TBR1. (FIG. 3I) We have integrated MVECs, which led to the expression of microvascular cytoarchitecture with the expression of VE-Cadherin to study BBB permeability using agents. Our cCerebral is highly compatible with studying complex CNS diseases like Alzheimer's, Parkinsons′, and many others. Scale bar 130 μm. The arrow indicates a specific marker expression.

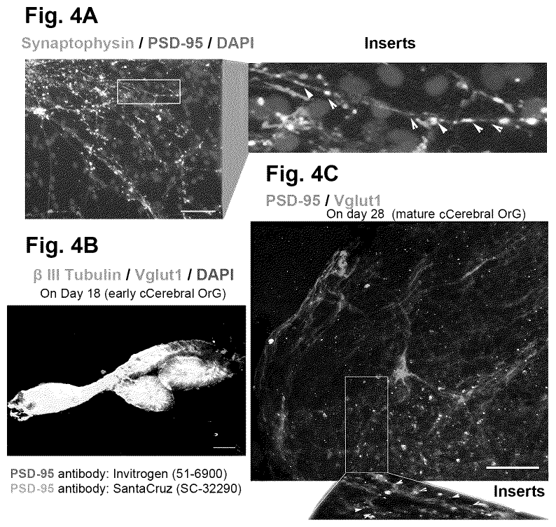

FIG. 4A-4C. Generation of functional glutamatergic excitatory cortical neurons. We have generated glutamatergic excitatory neurons expressing (FIG. 4A) postsynaptic density marker postsynaptic density-95 (PSD-95) and presynaptic marker synaptophysin. Inserts with arrowheads indicate the interaction of presynaptic and postsynaptic proteins on the axons, prove synaptic transmission of cCerebral neurons with an active action potential, (FIG. 4B) Our corticosphere (Csphere) at day 18 produces large numbers of buds in the gel-matrix domes with neuron-specific marker expression (III Tubulin and vesicular glutamate transporter 1 (Vglut1). During neurogenesis, Vglut1 started to express from as early as day 18. (FIG. 4C) In our cCerebral, highly expressed Vglut1 and PSD-95. Inserts with arrowheads showed PSD-95 puncta associated with Vglut1, which provides insights into the neuronal glutamatergic signaling in our cerebral OrGs usually observed in the human postnatal developmental maturation stage. Our cCerebral OrG is functional and represents highly active synaptic interaction, providing a model for studying neurodevelopmental or neurodegenerative diseases or sensitive drug screening platforms. Scale bar 130 μm. The arrowheads indicate a specific marker expression.

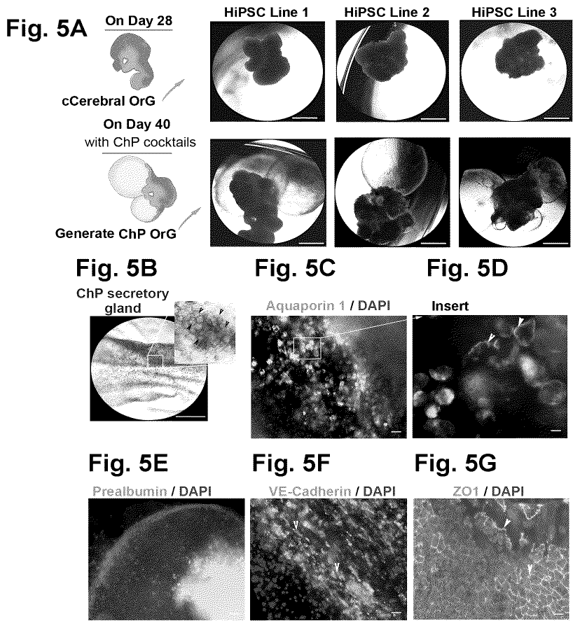

FIG. 5A-5G. Development of reproducible ChP OrG with their marker expressions. To generate ChP organoids, we have used OrGspheroids, which were initially treated with ChP growth cocktails containing 50% serum replacement. (FIG. 5A) The ChP OrG procedure has the same step as cCerebral OrG except that the initial growth media of OrGspheroids culture was different. ChP OrG needs longer to appear as typical ChP epithelium surrounding the fluid-filled cavity with CSF. We have observed that ChP-CSF organoids progressively mature over time, which takes up to 40 days or even more. We have successfully generated ChP OrG from three different HiPSC lines, and they are comparable to cCerebral OrG. On Day 40, mature ChP OrG produces a large amount of CSF and provides a protective barrier of blood CSF barrier (B-CSFb) similar to the human brain in vivo. Scale bar 1550 μm. (FIG. 5B) Giemsa staining showed the ChP secretory gland (arrow), which actively secretes CSF. Scale bar 1375 μm (FIG. 5C-5E) CSF markers aquaporin-1 and pre-albumin expression determined by ICC usually found these components in secreting CSF in vivo. Scale bar 70 and 130 μm respectively (FIG. 5F-5G) Endothelial (VE-Cadherin) and tight junction protein markers (ZO1) in ChP epithelium appear after day 40 representing B-CSFb. Scale bar 70 μm.

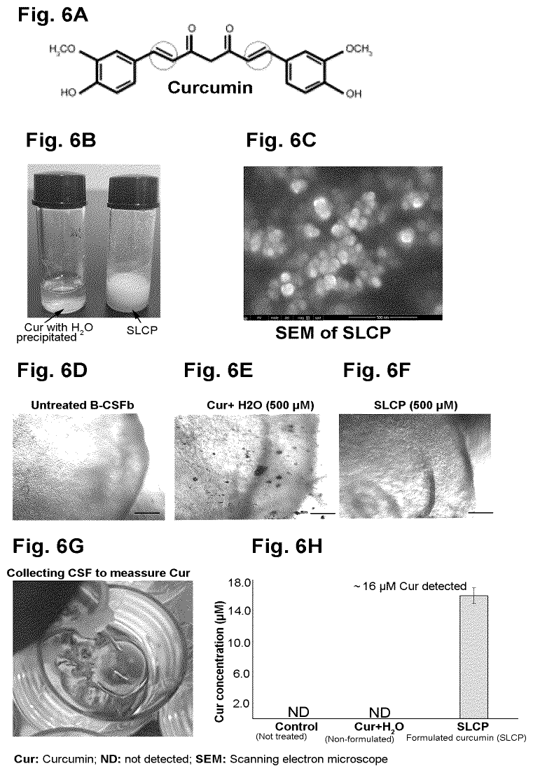

FIG. 6A-6H. ChP OrG efficiently screens drug permeability. (FIG. 6A) We have used curcumin (Cur), a natural polyphenolic antioxidant that can disaggregate Amyloid beta (AB) and tau. (FIG. 6B) We have prepared the Cur with H₂O and lipids to generate a solid lipid curcumin particle (SLCP). Curcumin precipitated on the bottom of the glass vial when prepared with H₂O (arrow), which is non-formulated Cur. (FIG. 6C) Solid lipid curcumin particle (SLCP)-formulated Cur was scanned by scanning electron microscope (SEM) and showed Cur nanoparticles (<100 nm). (FIG. 6D-6F) We added SLCP and non-formulated (H₂O dissolved) Cur to the blood CSF barrier brain OrG (ChP OrG) for 72 h and obtained images. (FIG. 6G) We then collected CSF via a syringe and processed it to measure optical density using a plate reader to determine whether SLCP Cur (500 μM) was permeable into CSF. (FIG. 6H) SLCP Cur was permeable into B-CSFb and obtained around 16 μM concentration, but non-formulated Cur precipitated on top of the B-CSF barrier (arrow) and was not detected in CSF. Our novel B-CSFb grown on an HT electrode plate is highly efficient in studying drug permeability on the HT scale and provides insight into using our ChP OrG to accelerate the drug development process for various CNS diseases. Scale bar 100 μm.

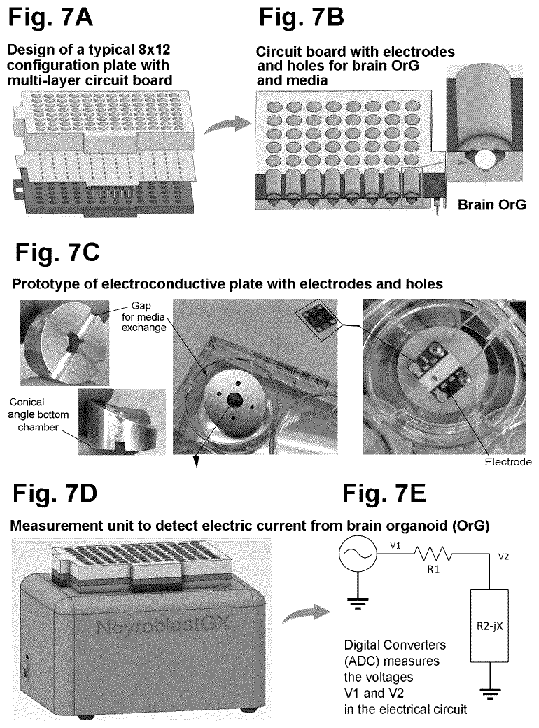

FIG. 7A-7E Rapid HT screening electrode plate for floating OrG. We have designed and developed a stepwise fabrication process for HT screening electrode plate for floating brain OrG (cCerebral and ChP OrG). It is unique to our brain OrG to control their size and shape and is easy to maintain to have results with higher sensitivity and specificity. (FIG. 7A) We have designed and developed a prototype electrode plate with a typical 8×12 configuration with multi-layer circuit boards in 96-well format. (FIG. 7B) The multi-layer circuit board contains measurement electrodes with holes for depositing the brain OrG and growth media to maintain floating OrG. (FIG. 7C) Cross-section of a prototype electrode on the well plate. In the center, a hole is for depositing a new brain OrG into the bottom part of the well and exchanging media, and the bottom is placed on the electrode to have a signal from a shallow depth of the sensor well with a size up to 5 mm. (FIG. 7D-7E) The design of the measurement unit consists of an Ethernet-enabled MCU that controls a Direct Digital Synthesizer that allows excitation signals to be generated with variable frequency and amplitude. This prototype is already used, and it provides a highly controlled MPS for OrG to grow and maintain without moving out from the electrode space to provide signals from specific OrG to provide an entire brain network rather than selective neurons. Thus, this new and novel HT screening tool provides novelty to electrode systems for screening new agents simultaneously and investigating diseases or new drugs more efficiently.

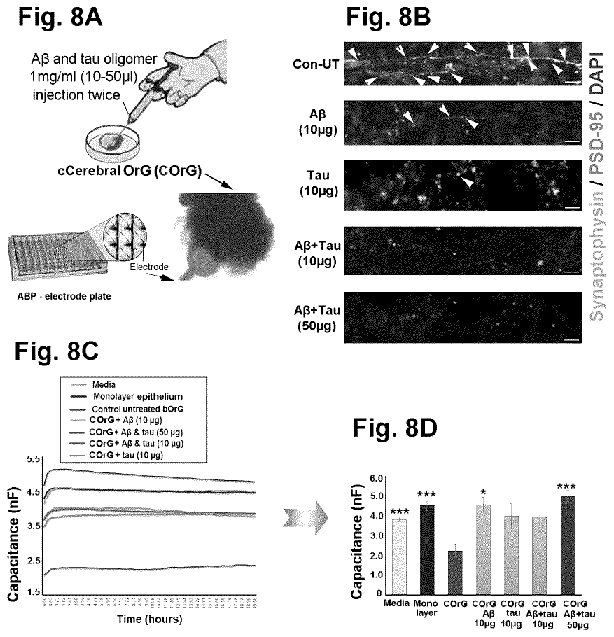

FIG. 8A-8D. Verify compatibility to screening NGL ready-to-use brain OrG in other screening platforms with multiple agents. (FIG. 8A) NGL cCerebral was injected twice with 10-50 μl (1 mg/ml) of Alzheimer's disease (AD) toxic proteins (AB and tau oligomers). (FIG. 8B) We found AB and tau together, especially 50 μg concentration, reduced in higher levels of pre- and post-synaptic density marker proteins (synaptophysin and PSD-95) compared to 10 μg concentrations using ICC. AB or tau alone was unable to significantly reduce synaptic proteins. (FIG. 8C) We compared synaptic loss with the electrode plate signal developed by Applied Biophysics (ABP). We have plated our cCerebral OrG onto ABP electrodes with gel-matrix twice coating to attach efficiently onto cCerebral OrG onto electrodes. Usually, marketed electrode attaching floating OrG tends to be challenging. OrG often slips or moves away from the electrode, thus needing twice or triple-coating layers with gels, providing a great insight into our floating electrode plate novelty compared to other marketed electrodes. After the cCerebral OrG attachment was maintained and grown on the electrode plate for one week and treated with or without the combination of AB and tau (10 and 50 μg). (FIG. 8D) We found Aβ and tau (50 μg) showed significant differences in electric current (nF, nanofarad) measurement using the voltage of 64000 Hz. AD toxic protein's electrical current of cCerebral OrG is comparable to synaptic loss and other biomarkers in AD/ADRD, which can be efficiently used for drug testing for any disease. *P<0.05 represents significant (n=5) and Dunn's multiple comparisons test.

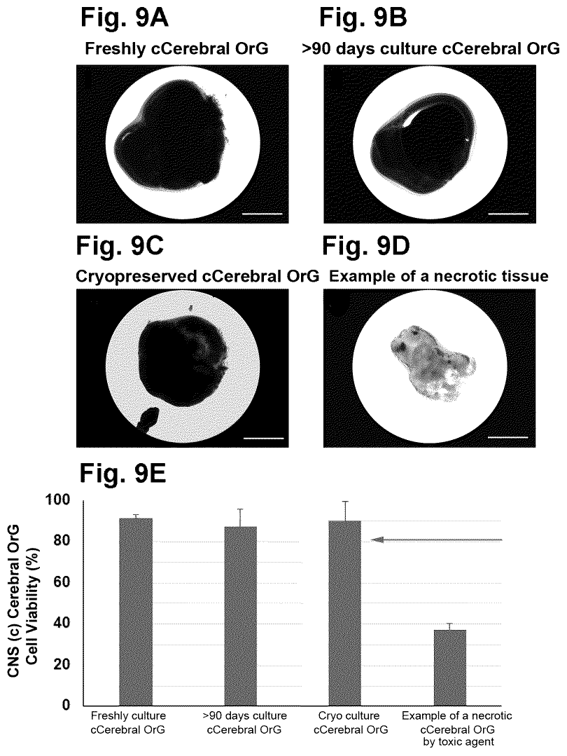

FIG. 9A-9E. Freshly and prolonged cultured brain OrG versus Cryo brain OrG viability and stability. The MTT [3-(4, 5-dimethylthiazol-2-yl)-2, 5-diphenyltetrazoliumbromide)] assay is used to measure cellular metabolic activity as an indicator of cell viability, proliferation, and cytotoxicity. (FIG. 9A-9D) To perform the MTT assay, we grew cCerebral OrG in 24 well plates for 28 days and prolonged culture kept for 90 days. We also used cryopreserved (Cryo) cCerebral OrG, which was thawed and immediately seeded using gel-matrix and maintained culture for 10 days. All the freshly prolonged and Cryo cultures were maintained with complete neural growth media. MTT assay was performed on (FIG. 9A) freshly, (FIG. 9B) prolonged, and (FIG. 9C) Cryo cCerebral OrG cultures, including (FIG. 9D) a necrotic cCerebral OrG tissue, for colorimetric comparison and demonstration of cCerebral OrG viability and stability. Viable cells with an active metabolism converted MTT (yellow color) into a purple-colored formazan product, which is highly visualized in an MTT solubilized solution that was further measured with a microplate reader with an absorbance near 570 nm. (FIG. 9E) MTT quantification of cCerebral OrG results showed our Cryo cCerebral OrG is highly viable (>80%) after the freeze-thaw cycle and comparable to freshly cultured OrG. We did not observe any necrotic core or the reduction of cCerebral OrG viability over 90 days, and culture remains viable at over 80%, as shown with an arrow. However, we used a necrotic tissue culture derived from the ingestion of toxic agents that provides easy visualization and comparison to provide insight into the stability of freshly or Cryo or prolonged cultured cerebral OrG compared to necrotic tissue. Thus, NGL cCerebral OrG culture is highly stable in freeze-thaw cycles or long cultures and can be used to HT screen via various agents for extended periods.

### EXAMPLES

### Example 1

### OrGspheroid Culture Protocol

Human-induced pluripotent stem cell (HiPSCs)-derived OrGspheroid generated from our custom-made HiPSCs (Creative Bioarray, Shirley, NY). HiPSCs were seeded 3×10⁶ cells per well of 6 well plates. Once cell confluence >90%, the cell is dissociated by gentle cell dissociation reagent (StemCell Tech) followed by plating of dissociated fragmented cells in serum replacement base (SRB) media consisting of knockout serum replacer (KSR, Invitrogen), DMEM/F12 (Invitrogen), glutamax (Invitrogen), non-essential amino acids (NEAA, Invitrogen), 100 μM of β-mercaptoethanol (Invitrogen), 10 μM of rock inhibitor, 10 ng/ML of BfGF and EGF and 5 μg/mL of heparin (Sigma-Aldrich). All the growth factors purchased from StemCell Tech. Cells were kept for 3 days until cell aggregates and became OrGspheroids, over 100 μm size, which has unique properties like stem cells to further differentiate into any three lineages and produce desired lineage-specific organoids development.

### Example 2

### cCerebral OrG In-Vitro Development Protocol

OrGspheroids were treated with complete neural induction media consisting of SRB media supplemented with 10 μM SB431542 (StemCell Tech or Cayman Chemical) and 1 μM dorsomorphin (StemCell Tech) for 7 days to induce neurogenesis. We gradually decreased SRB and increased neural complete growth (NCG) media during neural induction. NCG consists of proportionally balanced DMEM/F12 and neurobasal media, including 1× N-2 (Invitrogen), 1× B27 (Invitrogen), 100 μM NEAA, 100 μM of β-mercaptoethanol, 2 mM glutamax, 50 μg/mL ascorbic acid, 1× insulin-transferrin-selenium cocktails, 50 U ml⁻¹ penicillin and 50 mg ml⁻¹ streptomycin. After the neural induction, OrGspheroid's edge becomes bright and translucent, but the OrGspheroid core becomes dark with 400-500 μm sizes with highly enriched cortical progenitors called corticoshere (Csphere). During this stage, Csphere is ready to seed in the gel-matrix domes (Corning or Invitrogen, either gel) in a 24-well treated plate. In this stage, we also incorporated previously prepared HiPSCs-derived microglia to integrate with cerebral OrG. We found Csphere started to produce buds within two days after the gel-matrix domes. On Day (D) 15, the Csphere started to produce large numbers of buds with the maintenance of NCG media, and D18 buds matured and formed cerebral organoids (OrG). In this stage, cerebral OrG is ready to re-seed or cryopreserved. Our method provides quicker and easier robust cerebral OrG generation, which only takes 18 days for cortical patterning. However, integration with microglia and microvascular endothelial cells (MVECs) takes just 28 days compared to previously established methods, which usually require 60-90 days. 4, 5, 14, 15 To generate CNS (c) Cerebral OrG, we re-seed cerebral OrG on ultra-low attachment 24-well plates with gel-matrix with microvascular endothelial cells (MVECs) and regularly maintained with NCG media. On D20 cCerebral OrG was removed by pipetting from the plate attachment or release automatically and maintained in a floating condition with an orbital shaker with continuous rotation. On D28, the cCerebral OrG becomes large with a dark core up to 3 mm, integrating microglia and MVECs with mature cortical neuronal, glial, and microvascular cytoarchitecture visualized, as shown in FIG. 3A. This is the novel new tri-layer culturing to generate human CNS mimics cerebral. NGL cCerebral OrG integrates microglia and MVECs without affecting the outcome of various cortical layer's neurons. NGL cCerebral OrG can maintain culture for four weeks to appear in mature microvessels. Our novel CNS cell-enriched complex cerebral OrG is highly enriched with microglia, astrocytes, each layer of cortical neurons, excitatory glutamatergic synaptic neurons, and microvessels. Thus, integrating glial cells, MVECs, into cerebral OrG and producing cCerebral OrG combined with HT electrode recapitulates the CNS brain microenvironment to rapidly and efficiently study neurodevelopmental or neurodegenerative disorders.

### Example 3

### ChP OrG In-Vitro Development Protocol

To generate ChP OrG, we use methods similar to those used to generate cCerebral OrG, except for the composition of OrGspheroid SRB media, which contained 50% KSR, 0.1% lipid-rich albumin (AlbuMAX), including all other components as stated in Example 1. For ChP OrG, neural induction was up to 9 days. Neural induction medium was the same as Example 2. Further, neural-induced OrGspheroid (>500 μm) was seeded with gel-matrix domes with a volume of OrGspheroid to MvECs at a 1:20 ratio. After that, OrGspheroid in the gel-matrix domes was in the attachment condition for a week. Further, gel-matrix coated OrGspheroids were released in floating NCG media with a rotation mode using an orbital shaker for up to 40 days to appear ChP OrG with a large fluid-filled cavity with the expression of ChP epithelium and CSF protein markers (FIG. 5A-5G). Our basic NCG media was the same for cCerebral and ChP OrG, which provides unique nutrition for CNS cells that differ from the recently published method requiring growth factors bone morphogenetic protein (BMP) 4 and others. & We have generated the NCG unique to brain nutrition. We can maintain various CNS cells without interfering with or inhibiting the growth of desired CNS neurons, glial cells, or any other required cells, providing novelty to our methods. Both OrGs are crucial brain OrGs and can be used to study various agents' permeability to CSF or blood-brain barrier mimic microvessels. Brain OrG (cCerebral and ChP OrG) are highly enriched with CNS cells, specifically cerebral, ChP, and secreting CSF. The blood CSF barrier or cCerebral can grow or be maintained in an HT-based electrode plate with floating conditions to obtain and monitor full brain activity with or without various agents to study or understand disease pathology.

### Example 4

### Fluorescent Imaging and Giemsa Staining

ICC is a common cellular protein or antigen-detecting and visualization technique that can recognize the target of interest or specific marker expression in cells via quantification imaging. The antibody is directly or indirectly linked to a reporter, such as a fluorophore or an enzyme. We performed ICC as a previously established method developed. 16, 17 Briefly, for ICC, we have grown brain OrG on coverslips coated or directly on the 24-well plates. Then, cells were washed with PBS (phosphate-buffered saline) and fixed with 4% paraformaldehyde (PFA, Thermo Scientific). After additional washes in PBS, the cells were permeabilized in 0.1% Triton X-100 (Sigma-Aldrich), followed by blocking with 5% BSA (bovine serum albumin) in PBS containing 10% normal goat serum (NGS, Thermofisher Scientific). Next, the cells were incubated overnight with primary antibodies diluted in a blocking solution at 4° C. with an appropriate dilution indicated by the manufacturer. The next day, the cells were washed three times with washing buffer (1×PBS containing 0.05% Tween 20 and 18 NGS). After that, brain OrG was incubated for 2 h at 25° C. with a fluorescent secondary antibody (Life Technologies). OrGs were further washed, and the coverslips with the cells were placed onto slides with a Fluoromount-G mounting medium containing 4′, 6-diamidino-2-phenylindole (DAPI-FG, Southern Biotech), but OrG without coverslips were incubated with DAPI dye (Thermo Scientific) directly on the plate. We performed ICC in various stages of OrG to determine their marker expression, including day 18, to observe early cortical neurogenesis (bud formation), mature cerebral with glial cells, layer-specific cortical neurons, excitatory synapses, and glutamatergic neurons at day 28 (FIG. 2A-4C). All antibodies were purchased from BD Biosciences or Invitrogen. NGL lab-produced cCerebral OrG followed a similar developmental pathway in the developing human brain^{14, 18}, including CSF secreting ChP OrG⁸. ChP OrG epithelium is a cuboidal cell monolayer, producing most of the CSF. ChP's cuboidal cell monolayer can easily be visualized by Giemsa staining (FIG. 5A-5G). The Giemsa stain is a differential stain that includes a combination of eosin dye, methylene blue, and azure in its composition. It binds specifically to the phosphate groups of DNA. Each component of Giemsa stain can stain fundamental components of cells such as cytoplasm, cellular granules, and nucleus. We used a ready-to-use Wright-Giemsa stain solution (Volu-Sol, Salt Lake City, Utah, diluted 1:1 in deionized water (DH₂O). We further applied 1 mL of diluted Giemsa staining solution on the ChP OrG plate, followed by the 5 min incubation at room temperature. After that, the ChP stain OrG was washed five times using DH₂O and further air dried. We have acquired an image immediately to capture e ChP epithelial cytoarchitecture. Our Giemsa staining demonstrated the CSF-secreting ChP cuboidal cell monolayer morphogenesis (FIG. 5A-5G).

### Example 5

### Ht Sensing Arrays in Combination with Brain Orgs for Screening Agents

To determine HT screening various agents, we designed and developed a next-generation HT electrode plate where brain OrG can grow in floating conditions with electrode chambers that can control OrG size without batch-to-batch variations. To develop these electrode sensor chambers (FIG. 7A-7B), we have designed and developed a typical 8×12 configuration that allows 96 samples to be measured and can be upgraded to 384 wells. Three essential components make up the well plate. The top block is a bio-compatible plastic with 96 wells. These wells are simple cylindrical shapes up to 7 mm in diameter and 12 mm in depth. The second component is a multi-layer circuit board containing measurement electrodes and holes for depositing the brain OrGs and growth media. The bottom third component also has 96 wells, but these are designed with a shallow depth (˜5 mm) and conical shape that allow Cspheres to grow at the center of the well and make reliable contact with the electrodes. FIG. 7C shows a prototype of the well plate and a close-up view of one well. The center hole of the bottom chamber is for depositing a new Csphere, and additional holes on the side are for flowing and exchanging growth media. As the Csphere grows in the lower well, it will fill the lower cell and contact the electrodes etched on the bottom side of the circuit board. At this point, the fully-grown brain OrG from the Csphere is confined to the lower well, so electrical contact remains reliable. In addition, we can use ABP's or any other commercial MSU for our HT electrical measurement. Our brain OrG is highly compatible with ABP or any other commercial electrode plate for screening various agents. Our HT electrode plate has two electrode probes to contact the brain OrG from the top and bottom. Thus, our HT electrode plate bottom and top electrodes will accurately detect full brain electric current over the measurement period. However, we have also designed the MSU, which consists of an Ethernet-enabled MCU that controls a Direct Digital Synthesizer to generate excitation signals with variable frequency and amplitude (FIG. 7D-7E). Further, Digital Converters measure the voltages V1 and V2 at two points in the electrical circuit. These converters will sample at four times the excitation frequency, thus allowing both the amplitude and phase of the signal at V1 to V2 be measured with an algorithm.

We also tested our ORGs with ABPs to understand which can be combined with their electrode plate and provide valuable insight from drugs, chemicals, or various agents treated samples. To do this, NGL cCerebral was plated in ABP's ECIS plate using gel-matrix and maintained for a week. After one week of culture, cCerebral OrG was injected twice with 10-50 μl (1 mg/ml) of AB and tau oligomers and found AB and tau together, especially 50 μg concentration, reduced pre- and post-synaptic density marker proteins (synaptophysin and PSD-95) substantially compared to 10 μg concentrations a shown in FIG. 8B. AB or tau alone treatment was unable to significantly reduce synaptic proteins. We compared synaptic loss with ABP's electrode plate signal. We have plated our cCerebral OrG onto ABP electrodes with gel-matrix twice coating to attach efficiently onto cCerebral OrG onto electrodes. Usually, marketed electrode attaching floating OrG tends to be challenging. OrG often moves away from the electrode, thus needing twice or triple-coating layers with gels, providing novelty to our floating electrode plate compared to other marketed electrodes. After the cCerebral OrG attachment was maintained and grown on the electrode plate for one week and treated with or without the combination of Aβ and tau (10 and 50 μg) and found Aβ and tau (50 μg) showed significant differences in electric current (nF, nanofarad) measurement using the voltage of 64000 Hz. AD toxic protein's electrical current of cCerebral OrG is comparable to synaptic loss and other biomarkers in AD/ADRD, which can be efficiently used for drug testing for any disease. *P<0.05 represents significant (n=5). We have analyzed data using Dunn's multiple comparisons test using GraphPad Prism (San Diego, CA).

### Example 6

### In-Vitro MTT Cell Viability Assay

We determined cCerebral OrG viability after the freeze-thaw cycle and compared with freshly and prolonged culture cCerebral OrG using a colorimetric assay to evaluate cell metabolic activity with an MTT [3-(4, 5-dimethylthiazol-2-yl)-2, 5-diphenyltetrazolium bromide] assay (FIG. 9A-9D), following the manufacturer's instructions for the assay (MilliporeSigma). To perform the reported established MTT assay¹³, we grew cCerebral OrG in 24-well plates for 28 days and prolonged culture kept for 90 days. We also used cryopreserved (Cryo) cCerebral OrG, which was thawed and immediately seeded using gel-matrix and maintained culture for 10 days. To observe OrG viability and stability, we have added 1 mg/mL of MTT reagent to each -well containing cCerebral OrG, followed by the 2 h incubation at 37° C. Further, OrG was washed with sterile PBS, and dimethyl sulfoxide (DMSO) was added to each well and mixed. After the 10 min incubation, DMSO was absorbed the MTT and run for a plate reader (SpectraMax iD3 Plate Reader, Molecular Devices, VWR International, PA) with the optical absorption density (OD) of 570 nm to determine the amount of MTT binding in the viable OrG cell. This method determined our cCerebral OrG's viability and stability after the freeze-thaw cycle or culture over 90 days. We calculated the amount of MTT in each well using the following formula:

$$Cell Viability ( \% ) = \frac{OD 570 treated c Cerebral OrG}{OD 570 control c Cerebral OrG} \times 100$$

### REFERENCE CITED IN BACKGROUND, SUMMARY, AND EXAMPLES

1. Zhou J Q, Zeng L H, Li C T, et al. Brain organoids are new tool for drug screening of neurological diseases. Neural Regen Res. 2023; 18 (9): 1884-1889. doi: 10.4103/1673-5374.367983
2. Sun N, Meng X, Liu Y, Song D, Jiang C, Cai J. Applications of brain organoids in neurodevelopment and neurological diseases. J Biomed Sci. 2021; 28 (1): 1-16. doi: 10.1186/s12929-021-00728-4
3. Wang Z, Wang S N, Xu T Y, Miao Z W, Su D F, Miao C Y. Organoid technology for brain and therapeutics research. CNS Neurosci Ther. 2017; 23 (10): 771-778. doi: 10.1111/cns.12754
4. Lancaster M A, Corsini N S, Wolfinger S, et al. Guided self-organization and cortical plate formation in human brain organoids. Nat Biotechnol. 2017; 35 (7): 659-666. doi: 10.1038/nbt.3906
5. Lancaster M A, Knoblich J A. Generation of cerebral organoids from human pluripotent stem cells. Nat Protoc. 2014; 9 (10): 2329-2340. doi: 10.1038/nprot.2014.158
6. Lancaster M A, Renner M, Martin C A, et al. Cerebral organoids model human brain development and microcephaly. Nature. 2013; 501 (7467): 373-379. doi: 10.1038/nature12517
7. Pellegrini L, Lancaster M A. Breaking the barrier: In vitro models to study choroid plexus development. Curr Opin Cell Biol. 2021; 73:41-49. doi: 10.1016/j.ceb.2021.05.005
8. Pellegrini L, Bonfio C, Chadwick J, Begum F, Skehel M, Lancaster M A. Human CNS barrier-forming organoids with cerebrospinal fluid production. Science (80-). 2020; 369 (6500). doi: 10.1126/science.aaz5626
9. Kashyap G, Bapat D, Das D, et al. Synapse loss and progress of Alzheimer's disease-A network model. Sci Rep. 2019; 9 (1): 1-9. doi: 10.1038/s41598-019-43076-y
10. Perez F P, Bose D, Maloney B, Nho K, Shah K, Lahiri D K. Late-onset Alzheimer's disease, heating up and foxed by several proteins: Pathomolecular effects of the aging process. J Alzheimer's Dis. 2014; 40 (1): 1-17. doi: 10.3233/JAD-131544
11. Qian X, Song H, Ming G L. Brain organoids: Advances, applications and challenges. Dev. 2019; 146 (8). doi: 10.1242/dev.166074
12. Giandomenico S L, Sutcliffe M, Lancaster M A. Generation and long-term culture of advanced cerebral organoids for studying later stages of neural development. Nat Protoc. 2021; 16 (2): 579-602. doi: 10.1038/s41596-020-00433-w
13. Grabinger T, Luks L, Kostadinova F, et al. Ex vivo culture of intestinal crypt organoids as a model system for assessing cell death induction in intestinal epithelial cells and enteropathy. Cell Death Dis. 2014; 5 (5): e1228-10. doi: 10.1038/cddis.2014.183
14. Anastasaki C, Wilson A F, Chen A S, Wegscheid M L, Gutmann D H. Generation of human induced pluripotent stem cell-derived cerebral organoids for cellular and molecular characterization. STAR Protoc. 2022; 3 (1): 101173. doi: 10.1016/j.xpro.2022.101173
15. Sivitilli A A, Gosio J T, Ghoshal B, et al. Robust production of uniform human cerebral organoids from pluripotent stem cells. Life Sci Alliance. 2020; 3 (5): 1-10. doi: 10.26508/1sa.202000707
16. Begum A N, Guoynes C, Cho J, Hao J, Lutfy K, Hong Y. Rapid generation of sub-type, region-specific neurons and neural networks from human pluripotent stem cell-derived neurospheres. Stem Cell Res. Published online 2015. doi: 10.1016/j.scr.2015.10.014
17. Begum A N, Aguilar J S, Elias L, Hong Y. Silver nanoparticles exhibit coating and dose-dependent neurotoxicity in glutamatergic neurons derived from human embryonic stem cells. Neurotoxicology. Published online 2016. doi: 10.1016/j.neuro.2016.08.015
18. Hopkins H K, Traverse E M, Barr K L. Methodologies for generating brain organoids to model viral pathogenesis in the CNS. Pathogens. 2021; 10 (11). doi: 10.3390/pathogens10111510

1. A method of screening and/or evaluating the effects of one or more agents on the brain, said method comprising:
exposing one or more agents to a multiwell electrode plate wherein wells in said plate contain brain organoid cells;
obtaining electrical signals from the electrode plate after the application of said one or more agents and
analyzing the electrical signals to evaluate the effect of said result of one or more agents on said brain organoid cells.

2. The method of claim 1, wherein:
said multiwell plate is a high throughput plate wherein each well comprises multiple working electrodes with multiple counters electrodes and one or more organoids;
each of said brain organoids comprises multiple functional tissues and/or components of an organ of a subject.

3. The method, according to claim 2, wherein said brain organoid is a fully functional specialized brain organoid grown from human induced pluripotent stem cells (HiPSCs).

4. The method, according to claim 1, wherein the electrical signals are determined by multiple frequencies from 62.5 to 64000 Hz.

5. The method, according to claim 3, wherein said one or more agents are placed in contact with the HiPSCs to grow the OrG with one or more conditions.

6. The method according to claim 1, wherein the evaluation is conducted to evaluate the effect of said agents on at least one condition selected from the group consisting of neurodegenerative disorders (e.g., cerebral amyloid angiopathy, dementia, traumatic brain injury, Alzheimer's, and Parkinson's disease).

7. The method, according to claim 3, wherein said brain organoid is grown from HiPSCs of a specific subject to screen and/or evaluate the effects of one or more agents on the subject.

8. The method, according to claim 7, wherein said brain organoids are floating in a nutrient media in neural media in the wells of said multiwell electrode plate, thereby providing a high throughput screening method for disease or personalized drug screening with high specificity.

9. The method, according to claim 1, wherein said one or more agents is at least one member selected from the group consisting of a therapeutic agent, drug, toxic agent, and pathogens.

10. The method according to claim 9, wherein said one or more agents is at least one member selected from the group consisting of LPS endotoxin, amyloid-beta (AB), tau oligomer, a chemical, a drug, a microbe, a bacteria, a virus (e.g., SARS-COV-2 virus such as COVID-19), gut microbiota.

11. A three-dimensional brain organoid comprising cerebral and/or choroid plexus (ChP) cells in combination with microglial and microvascular endothelial cells.

12. The brain organoid, according to claim 11, comprises a tri-layered structure of central nervous system cells.

13. The brain organoid, according to claim 11, wherein said cerebral and/or ChP cells are produced from HiPSCs co-cultured with microglial and microvascular endothelial cells.

14. The brain organoid, according to claim 11, wherein said brain organoid is floating in a neural media.

15. A system for screening the response of brain organoids to an active agent comprising:
a multi-well electrode conductive plate; and
a brain organoid, according to claim 11, contained within said wells.

16. A method for preparing a brain organoid comprising:
a) culturing HiPSCs in a first culture media to produce 3-dimensional organoid spheroid (OrGspheroids) cells;
b) treating said 3-dimensional OrGspheroid cells with neural induction media to produce corticospheres;
c) seeding said corticospheres on gel matrix domes and with microglia and microvascular endothelial cells to produce CNS cell enriched cerebral organoids as a human postnatal brain; and
e) culturing said cerebral organoid in neural complete growth media to provide a complete cerebral organoid.

17. A method for preparing a brain organoid comprising:
a) culturing HiPSCs in a first culture media to produce 3-dimensional organoid spheroid (OrGspheroids) cells;
b) treating said 3-dimensional OrGspheroid cells with neural induction media to produce corticospheres;
c) seeding said corticospheres on gel matrix domes and with microvascular endothelial cells to produce choroid plexus organoids; and
e) Culturing said cerebral organoids in neural complete growth media to provide a complete chloroid plexus (ChP) organoid.

18. A brain organoid prepared by the method of claim 16.
